(19) United States
(12) Patent Application Publication
(10) Pub. No.: US 2011/0200545 A1
(43) Pub. Date: Aug. 18, 2011

(54) DEODORANT COMPOSITION AND METHODS FOR USE THEREOF

(76) Inventor: Nyangenya Maniga, Gilbert, AZ (US)

(21) Appl. No.: 13/025,417

(22) Filed: Feb. 11, 2011

Related U.S. Application Data
(60) Provisional application No. 61/304,461, filed on Feb. 14, 2010.
(60) Provisional application No. 61/306,519, filed on Feb. 21, 2010.

(51) Int. Cl. A61K 8/44; A61Q 15/00; C11D 3/26

(52) U.S. Cl. 424/65; 510/130; 510/158; 510/319

(57) ABSTRACT

The invention comprises a deodorant composition for the treatment of fibers, hair, and/or skin. Preferred embodiments include compositions containing one or more of active ingredients L-arginine, acetylsalicyclic acid, and salicyclic acid adjusted to a pH in the range of 4.0 to 6.0. Preferred compositions include: a carrier, such as water; a moisturizer, such as propylene glycol; one or more of the active ingredients; along with optional preservatives, herbal extract, and skin protectants. Application of a composition for the treatment of odor forming microbes with a pH of about skin does not disrupt normal skin functioning.

### CROSS-REFERENCES TO RELATED APPLICATIONS

This application claims:
the benefit of U.S. provisional patent application No. 61/304,461 filed Feb. 14, 2010; and
the benefit of U.S. provisional patent application No. 61/306,519 filed Feb. 21, 2010,
all of which are incorporated herein in their entirety by this reference thereto.

### BACKGROUND OF THE INVENTION

1. Field of the Invention

This invention relates generally to a deodorant and methods for the application thereof.

2. Discussion of the Prior Art

### Body Odor

Body odor or bromhidrosis is also referred to as osmidrosis and ozochrotia, which is also known as apocrine bromhidrosis, bromidrosis, fetid sweat, body smell, malodorous sweating, and osmidrosis, which is the smell of bacteria growing on the body.

### Body Odor Prevention/Treatment

An individual's body odor is influenced by diet, gender, genetics, health, and medication. Although body odor is affected by diet, the primary approaches to reduction of body odor is through hygiene practices and with medication. Particularly, reduction of body odor is achieved through the use of deodorants; antiperspirants; disinfectants, such as triclosan; special soaps; foams with antiseptic plant extracts, such as ribwort and liquorice; and use of chlorophyllin.

Aluminum is a common component of antiperspirants. Aluminium in antiperspirants is implicated as a risk factor for both breast cancer and Alzheimer's disease. Further, aluminium has no known function in living cells and presents some toxic effects in elevated concentrations. Hence, control of body odor without the use of aluminum is beneficial.

Compounds exist for the treatment of body odor, such as deodorants, antiperspirants, and bath soaps. However, these compounds are different in chemical composition from the present invention. Further, the fact that body odor is still a serious condition is ample evidence that a more effective remedy is needed.

Patents related to the current invention are summarized here.

### Anti-Bacterial/Anti-Microbial/Germicide

D. Cox, et. al., “Cosmetic Compositions”, U.S. Pat. No. 7,510,704 B2 (Mar. 31, 2009) describe the used of a deodorant having an active ingredient of 4-hydroxy-3-methoxybenzyl alcohol as an effective, sub-lethal inhibitor of selected corynebacteria.

M. Horino, “Sebum-Adsorbent Powder and Use Thereof”, U.S. Pat. No. 6,730,309 B2 (May 4, 2004) describes the use of a core powder, a hydroxyapatite layered on the core, and a zinc layered on the hydroxyapatite, where the powder is used in adsorbing, solidifying, or congealing sebum components or odor components.

C. Schroeder, et. al., “Use of Nanoscalar Antimicrobial Active Ingredients in Body Deodorants”, U.S. Pat. No. 6,716,438 B1 (Apr. 6, 2004) describe a deodorant composition and method of making and using where the deodorant contains nanoscale particles containing one or more antimicrobial agents having a diameter of five to five hundred nanometers.

G. Holzner, “Antimicrobial Perfume Compositions”, U.S. Pat. No. 6,479,456 B1 (Nov. 12, 2002) describes an antimicrobial perfume composition having antimicrobial activity of at least eighty percent and an active ingredient of grapefruit-pip, a fumitory extract, a fumaric acid, or an ester of fumaric acid or lactic acid.

G. Acuna, et. al., “Fragrance Precursor Compounds for Preventing Human Malodor”, U.S. Pat. No. 5,925,339 (Jun. 20, 1999) describe the use of a compound having formula X—(Z)ₙ—CO—NH—CH—(COO—Y)—CH₂CH₂CONH₂ where X, Y, and Z are substituents and the formula is used for the prevention of human malodor.

M. Rockl, et. al., “Deodorizing Cosmetic Compositions”, U.S. Pat. No. 5,690,919 (Nov. 25, 1997) describe a mixture of dodecanoic acid, lauric acid, at least one other fatty acid, and glycerol monocaprylate or glycerol monocaprate in a pH range below eight as an active principle for the selective reduction of coryneform bacteria.

S. Mori, et. al., “Bath Medicine Composition and its Use in Inhibiting the Generation of Body Odor”, U.S. Pat. No. 5,665,742 (Sep. 9, 1997) describe the use of a bath medicine composition containing at least one germicide selected from the group consisting of: phenolic germicides, cationic germicides, and trichlorocarbanilide and a carbon dioxide generating component.

D. Rase, et. al., “Topical Antimicrobial Composition and Uses”, U.S. Pat. No. 5,169,631 (Dec. 8, 1992) describe a topical antimicrobial composition including microcapsules containing an antimicrobial agent, where the wall of the microcapsule is formed of collagen and glycosaminoglycan cross-linked with a cross-linking agent.

B. Ekenstam, et. al., “Germicidal Composition”, U.S. Pat. No. 4,557,935 (Dec. 10, 1985) describe a germicidal composition for application on skin consisting of an aqueous suspension of hydrophilic crystals of 1-monolaurin and preferably also 1-monomyristin in a quantity of twenty to thirty percent by weight and hydrogen peroxide in a quantity of two-tenths to five percent by weight.

### Deodorant/Perfumery

B. Buford, et. al., “Deodorant Pad System”, U.S. Pat. No. 6,743,418 B1 (Jun. 1, 2004) describe a deodorant pad immersed in a solution of baby powder, baking soda, deodorant, and water.

E. Frerot, et. al., “Compounds Derived From Menthol and Use as a Refreshing Agent”, U.S. Pat. No. 6,359,168 B1 (Mar. 19, 2002) describes the use of an isomer of 3-menthyl-3,6-dioxaheptanoate as a refreshing agent or in perfumery.

K. Schulte-Elte, et. al., “Perfuming Ingredients of Woody, Fruity Odor”, U.S. Pat. No. 6,008,186 (Dec. 28, 1999) describes the use of a 3-methoxy-7,7-dimethyl-10-methylene-bicyclo[4.3.1]decane or 7,7-dimethyl-10-methylene-bicyclo[4.3.1]dec-3-yl acetate.

W. Lee, et. al., “Cosmetic Cream Composition Containing Silicone Gel Material”, U.S. Pat. No. 5,919,437 (Jul. 6, 1999) describe a solid deodorant active composition bound with a silicone gel material, where the silicone material includes a volatile silicone material and an organopolysiloxane material.

I. Kawase, “Funeral Deodorant”, U.S. Pat. No. 5,895,625 (Apr. 20, 1999) describes a funeral deodorant for application to a dead body containing pyroligneous liquid.

L. Kilpatrick-Liverman, et. al., “Clear Cosmetic Stick Composition Containing Sucrose Esters and Method of Use”, U.S. Pat. No. 5,776,475 (Jul. 7, 1998) describe a clear, soap-gelled deodorant containing alcohol, water, at least one sucrose ester, and an active ingredient.

M. Shevade, et. al., “Clear Cosmetic Stick Composition Containing a Combination of Anionic and Non-Ionic Surfactants”, U.S. Pat. No. 5,681,552 (Oct. 28, 1997) describe a clear, soap gelled deodorant containing alcohol; water; and a combination of an anionic surfactant, such as sodium laureth-13 carboxylate; and a non-ionic surfactant, such as Ceteareth-55.

L. Kilpatrick-Liverman, et. al., “Clear Cosmetic Stick Composition”, U.S. Pat. No. 5,639,463 (Jun. 17, 1997) describe a cosmetic stick composition gelled with a soap gelling agent, alcohol, water, alkoxylate materials, and/or dimethicone copolyols.

R. Abad, “Therapeutic and Cosmetic Composition for the Treatment of Skin”, U.S. Pat. No. 5,538,740 (Jul. 23, 1996) describes the isolation of an active material from gastropoda, which is one-tenth to thirty percent a mixture of amino acids and atoxic substances used as a deodorant.

R. Kasat, et. al., “Transparent Clear Cosmetic Stick Composition Containing Sodium Salts of Methyl Carboxyl Derivatives of Ethoxylated Lauryl Alcohol”, U.S. Pat. No. 5,458,880 (Oct. 17, 1995), describe transparent, clear cosmetic stick compositions of the type gelled with sodium salts of saturated fatty acids that contain an alcohol. The composition contains sodium salts of methyl carboxy derivatives of ethoxylated lauryl alcohol to improve transparency and a gelling agent of sodium salts of fatty acids with carbon chain lengths of C₁₂ to C₂₂ along with an active deodorant material.

D. Malka, “Powdered Body Deodorant Composition”, U.S. Pat. No. 5,453,266 (Sep. 26, 1995) describes a powdered body deodorant composition containing alum, acacia gum, an inert powder, and zinc oxide.

R. Kasat, et. al., “Transparent Clear Stick Composition”, U.S. Pat. No. 5,424,070 (Jun. 13, 1995) describes a clear, transparent cosmetic solid stick composition containing an alcohol and water gelled with a soap. The composition also contains an eumulgin compound to provide clarity and transparency and both sodium chloride and stearyl alcohol to increase the melting point of the composition.

C. Santa Ana, “Disposable Body Deodorant Pad and Deodorant Preparation Thereof”, U.S. Pat. No. 5,403,588 (Apr. 4, 1995) describes a body deodorant composition of seventy percent isopropyl alcohol, acetone, and aluminum chloride, which provides improved cleansing and deodorizing effects.

M. Barr, et. al., “Enhanced Efficacy, Long-Lasting Fragrance Composition, and Deodorant Composition, for Masking Malodor, Containing the Fragrance Composition”, U.S. Pat. No. 5,380,707 (Jan. 10, 1995) describe a fragrance composition having enhanced efficacy for masking malodor for extended periods of time to be used in a deodorant composition containing acetyl hexamethyl tetralin.

D. Harris, et. al., “Liquid Crystal Deodorant”, U.S. Pat. No. 5,348,735 (Sep. 20, 1994) describe a liquid crystal deodorant produced by dissolving a powdered crystalline double sulfate of aluminum in a saturated solution of water or a combination of water and alcohol where the alcohol is preferably denatured alcohol.

J. Melanson, et. al., “Deodorant Compositions Containing Specific Piroctone Salts and Perfumes”, U.S. Pat. No. 4,906,454 (Mar. 6, 1990) describe deodorant compositions including piroctone acid, its nonprimary olamine salts and combinations thereof along with a perfume containing high concentration of aldehydes and/or ketones.

H. Meter, “Deodorant”, U.S. Pat. No. 4,883,651 (Nov. 28, 1989) describes a deodorant containing an extract of coffea arabinensis optionally combined with thea simensis, uritica, and/or knotgrass.

B. Bews, et. al., “Deodorant Compositions”, U.S. Pat. No. 4,708,863 (Nov. 24, 1987) describe a deodorant composition including an active ingredient of zinc carbonate and less than eighty percent water by weight.

B. Bews, et. al., “Deodorant Compositions”, U.S. Pat. No. 4,659,560 (Apr. 21, 1987) describe a deodorant composition including an active ingredient of an oxide, hydroxide, a carbonate of magnesium, a carbonate of lanthanum or a mixture thereof and less than eighty percent water by weight.

### Encapsulation

A. Shefer, et. al., “Multi Component Controlled Release System for Anhydrous Cosmetic Compositions”, U.S. Pat. No. 7,115,282 B2 (Oct. 3, 2006) describe a controlled release system incorporated into anhydrous cosmetic formulations to provide moisture triggered release and prolonged release of encapsulated active ingredients.

J. Burger, et. al., “Moisture and Oxygen Stable Composition and a Process for Obtaining Said Composition”, U.S. Pat. No. 7,018,651 B2 (Mar. 28, 2006) describe a moisture and oxygen stable composition including inert core particles and a partial or complete coating thereon of a least one active compound, where the active compound is optionally a pharmaceutical.

D. Roux, et. al., “Active Principal Carriers Containing Non-Ionic Surfactants, and Uses Thereof Particularly in Food, Cosmetics and Pharmaceuticals”, U.S. Pat. No. 5,908,697 (Jun. 1, 1999) describe non-ionic surfactants where the surfactants consist of sucrose and fatty acid esters forming onion-structured microcapsules encapsulating an active material.

### Cosmetic Composition

E. Bollens, et. al., “Cosmetic Compositions Containing Hydrofluorocarbon Compounds”, U.S. Pat. No. 5,705,165 (Jan. 6, 1998) describe a cosmetic composition containing hydrofluorocarbon compounds, sulfoxide compounds, and sulfone compounds.

pH

O. Yamashita, et. al., “Deodorants”, U.S. Pat. No. 6,780,403 B1 (Aug. 24, 2004) describes a liquid deodorant suitable for deodorization of spaces and hair products, the deodorant composed of an organic dibasic acid having a pK₂ of 1.7 or more different from pK₁ and in the range of 6.0 to 8.0 with a salt from a group consisting of an alkali metal, an ammonium, a zinc, and an aluminum.

### Problem

There exists in the art a need for a treatment of fiber and/or body odor. There further exists a need for treatment of hair and/or skin with a preventative composition and method of application thereof that does not irritate the skin, peel skin, or alter the pH of skin and its corresponding functionality. Further, there exists a need for treatment of body odor without the use of aluminum.

### SUMMARY OF THE INVENTION

The invention comprises a deodorant composition for the treatment of fiber products, hair, and/or skin.

### DESCRIPTION OF THE FIGURES

FIG. 1 illustrates formation of a deodorant and/or fiber treatment composition; and

FIG. 2 illustrates various forms of L-arginine.

### DETAILED DESCRIPTION OF THE INVENTION

The invention comprises a composition and/or application thereof for preventing the formation of human malodor, an unpleasant odor, stench, and/or offensive odor. More particularly, the composition or treatment with the composition prevents odor resulting from bacterial action. Various embodiments of the composition are described, infra. In use, all or part of the composition is applied to a fiber and/or skin surface or as a bath medicine. In the case of application to the skin or as a bath medicine, the composition optionally enters into the skin and/or penetrates into a hair follicle and/or pore. The composition is optionally pH balanced so as to not irritate the skin or alter its functionality.

Bacteria present on the skin produce chemical compounds perceived as body odor. Particularly, propionobacteria, which thrive in the ducts of adolescent and adult sebaceous glands, are a genus of bacteria that live in and around the sweat glands, sebaceous glands, and other areas of the skin of humans. Propionobacteria breakdown some amino acids to create propionic acid or propanoic acid, which is associated with body odor and has a vinegar-like smell. Additionally, staphylococcus epidermidis bacteria produce isovaleric acid or 3-methyl butanoic acid, which is another source of body odor. These bacteria multiply rapidly in the presence of sweat, but sweat itself is almost completely odorless to humans.

In a first embodiment, a composition is described which impedes bacterial action creating odorous compounds. For example, the composition is optionally used to impede bacterial action on the body, in a bath, and/or on fiber, such as a garment being worn or cleaned. While the composition is described using body odor as an example, any of the formulations or compositions described herein are equally applicable to treatment and/or prevention of bacterially created odorous compounds and/or molecules in other environments, such as in or on a fibrous material or an article of clothing. Further, while the composition is described using body odor as an example, any of the formulations or compositions described herein are equally applicable as additives to any deodorant, antiperspirant, soap, bath additive, shower gel, skin cleaning product, clothing cleaning product, or cleaning product.

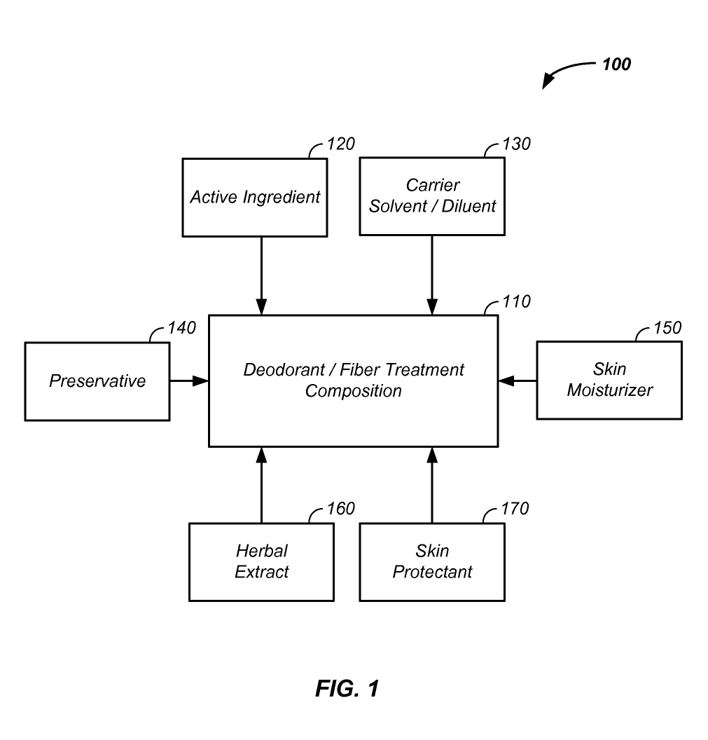

Referring now to FIG. 1, a deodorant formulation method 100 is illustrated. The deodorant 110 includes an active ingredient 120 and optionally includes one or more of a carrier 130, a preservative 140, a skin moisturizer 150, an herbal extract 160, and a skin protectant 170.

In one embodiment, a composition of the deodorant 110 is used for prevention of and/or treatment of odor formed by bacterial action. For example, the deodorant 110 impedes odor forming bacteria from multiplying, inactivates odoriferous bacteria, blocks odiferous mercaptans from forming by bacterial actions, and/or blocks formation of odorous sulfur acids by bacterial action. A preferred composition includes L-arginine with any of aspirin or acetylsalicyclic acid or salicyclic acid. Additional optional compositions include L-arginine with any of:
one or more hydroxyacids; and
a mixture of one or more hydroxyacids and aspirin.

In any of the compositions, water is an optional carrier or diluent 130. An additional optional composition element is a skin moisturizer 140, such as propylene glycol.

In another embodiment, the composition 110 includes: at least one amino acid and at least one of α-hydroxy acid, salicyclic acid, and acetyl salicyclic acid.

While any amino acid is optionally used, the amino acids of arginine, lysine, and/or histidine are preferred as they are of a basic pH and do not result in deproteinization of the aforementioned acids. The hydrogen ion is held in a complex with the amino acid. By not removing the proton, the acid is intact and more readily penetrates the skin. Additionally, the acid pH of the amino acids does not result in a free proton, which is otherwise corrosive to the skin. Lysine, histidine, and arginine are preferred, but any protein is optionally used and hydrolyzed. For example, collagen or protein is used as a composition component and is hydrolyzed in the composition to result in amino acids, which react as described herein with one or more of α-hydroxy acid, salicyclic acid, and acetyl salicyclic acid.

In yet another embodiment, the composition includes at least one amino acid; at least one of α-hydroxy acid, salicyclic acid, and acetyl salicyclic acid; and a base.

### Composition Ingredients

In yet another embodiment of the invention, the composition contains one and preferably both of L-arginine and acetylsalicyclic acid and at least one of or any combination of: water, aloe vera gel, propylene glycol, lecithin, lactic acid, an alpha-hydroxy acid, shea butter, a lipid, a lipid material, retinoic acid, ascorbic acid, xanthan gum, and/or hydroxypropylmethyl cellulose.

### L-Arginine

A first optional active ingredient, active material, or active element of the composition is L-arginine, an enantiomer of arginine, and/or arginine, with a concentration range of 0.1 to 40 percent. L-arginine is an essential amino acid, C₆H₁₄N₄O₂. The recommended daily intake of L-arginine is two grams per day. The free amino acid is also generated by hydrolysis or digestion of plant and/or animal protein. L-arginine is a vasodilator that increases blood flow, is a fat solubilizer, and cures wounds. The free amino acid increases insulin secretion and is converted to urea in the liver by arginase.

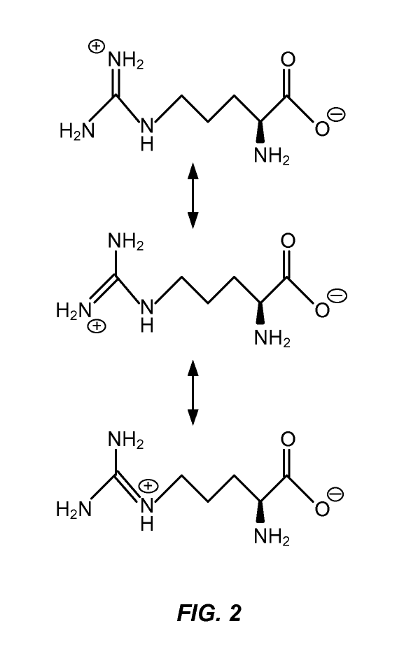

The amino acid side chain of arginine includes a 3-carbon aliphatic straight chain, the distal end of which is capped by a complex guanidinium group. With a pKₐ of 12.48, the guanidinium group is positively charged in neutral, acidic, and even most basic environments and thus imparts basic chemical properties to arginine. Because of the conjugation between the double bond and the nitrogen lone pairs, the positive charge is delocalized, enabling the formation of multiple hydrogen bonds, H-bonds. Referring now to FIG. 2, L-arginine is illustrated in various forms showing the delocalization of charge in the guanidinium group.

Arginine plays an important role in cell division, the healing of wounds, removing ammonia from the body, immune function, and the release of hormones. The benefits and functions attributed to oral supplementation of L-arginine include:
use as a precursor for the synthesis of nitric oxide, (NO);
reduction in healing time of injuries;
vasodilatation;
quickened repair time of damaged tissue; and
helping decrease blood pressure.

As a precursor, arginine is used in generation of nitric oxide, which relaxes blood vessels and is used when vasodilation is required.

In another embodiment, any of: L-arginine; arginine; lysine; glycine; histidine; a pH basic amino acid, such as an amino acid having at least one pKa greater than 7.0; and/or a fat solubilizer is used as an active ingredient in the formulation or composition used in the treatment of body odor. The percentage of L-arginine, lysine, and/or glycine alone or in combination is preferably about 0.1, 5, 10, 15, 20, 25, 30, 35, or 40 percent by volume or mass. Optionally, any source of amino acids is used, such as hydrolysed collagen or a protein hydrolysate. Optionally glucosamine and/or n-acetylglucosamine are used to dampen, adjust, or buffer the pH of the resulting composition.

### Acetylsalicyclic Acid

A second optional active ingredient, active material, or active element 120 of the composition 110 is acetylsalicyclic acid, with a concentration range of 0.1 to 40 percent. Acetylsalicyclic acid and its metabolized form of salicyclic acid each decrease inflammation. Addition of the acetyl group to salicyclic acid results in a molecule that is more non-polar than salicyclic acid. The less polar acetylsalicyclic acid is more readily absorbed into skin cells and more readily transports across a lipid membrane of a skin cell. Once inside the cell, the acetyl group is removed to form an active ingredient of salicyclic acid. Hence, acetylsalicyclic acid is a preferred constituent of the composition for treating body odor. However, an optional embodiment of the composition for treatment of bacterially created odorous molecules contains one or more of: salicyclic acid and/or any α-hydroxy acid, in the absence of acetylsalicyclic acid.

The inventor has determined that used by itself, acetylsalicyclic acid acts as a skin peeler, as the pKa is about 1. The low pKa results in an acidic solution having a pH of about 1.9, which damages skin and results in skin peeling in the hours and/or days following application to skin. Particularly, application of acetylsalicyclic acid in a composition having a pH of about 3.8 or less results in one or more of the detrimental effects of: an acid burn of the skin, inflammation, itching, sensitizing, dermatitis, and/or skin peeling. The inventor discovered that adjusting the pH of a composition used to treat and/or prevent body odor containing acetylsalicyclic acid or salicyclic acid to about 5.5 or about 4.0 to 6.5 reduces and/or eliminates the above described detrimental effects.

In another embodiment, any of acetylsalicyclic acid, salicyclic acid, and or α-hydroxy acid is used as an active ingredient in the formulation or composition used in the treatment and/or prevention of body odor. The percentage of acetylsalicyclic acid, salicyclic acid, and or α-hydroxy acid alone or in combination is preferably about 0.1, 5, 10, 15, 20, 25, 30, 35, or 40 percent by volume or mass.

### Optional Active Elements

L-arginine and acetylsalicyclic acid are preferred active ingredients, constituents, or elements of a composition or formulation for the treatment of bacterially created odorous by-products. Additional or substitute optional active elements are described herein. One optional active element is retinoic acid. A preferred concentration range of retinoic acid is 0 to 0.05 percent. Retinoic acid is an oxidized from of Vitamin A and contains only partial functionality of Vitamin A. Retinoic acid acts faster on body odor causing bacteria as the acid is more non-polar than Vitamin A and hence transports across, into, or through skin layers more efficiently. An additional optional active element is Vitamin A. Both retinoic acid and Vitamin A remove swelling and/or remove dead skin. Yet another optional active element is lactic acid, which is an α-hydroxy acid. Lactic acid is a preservative 140, which is optionally used in place of and/or in combination with aspirin and/or acetylsalicyclic acid in the composition. Still yet additional active elements include any of: lysine, histidine, hydrolysed collagen, and protein hydrolysate.

### Water

Water is an optional carrier, solvent, or diluent 130 of the composition. Water is a carrier and/or diluent. When used, the percentage of water is preferably about 20 to 99 percent and more preferably about 40, 50, 60, 61, 62, 63, 64, 65, 70, or 80 percent by volume or mass.

### Skin Moisturizers

Each of glycerin; any lipid material, such hyaluronan, hyaluronic acid, soybean oil or shea butter; any vegetable oil, such as coconut oil or palm oil; aloe vera gel; propylene glycol; lecithin; or a solubilizer are both (1) optional composition components and (2) perform as skin moisturizers 150. Hyaluronic acid is both a stabilizer and a moisturizer and can hold one thousand times its own weight in water, which is beneficial in a deodorant. Shea butter, lecithin, and/or a lipid material are optionally used independently or in combination to enhance penetration of the active material, such as L-arginine and/or acetylsalicyclic acid, into the skin. Propylene glycol additionally performs as a penetration enhancer and performs as a solubilizer for salicyclic acid and/or acetylsalicyclic acid. The percentage of aloe vera gel, propylene glycol, lecithin, shea butter, or a solubilizer alone or in combination is preferably about 0, 5, 10, 15, 20, 25, 30, 35, or 40 percent by volume or mass. Preferably, one or a combination of the moisturizers 150 are optionally present in the deodorant composition 110 from about zero to forty percent by weight.

### Skin Protectants

Each of glycerin and non-toxic salts are optionally included in the deodorant composition as skin protectants or skin protectors 170. A non-toxic salt preferably is composed of zinc salts or magnesium salts, such as magnesium sulfate, which if absorbed by the body is good for you, as opposed to salts of aluminum and/or silver. For example, aluminum chloride, potassium aluminum, aluminum salts, and/or a form of silver are optionally included but are preferably not included in the deodorant composition 110. By contrast, the preferred salts of zinc and magnesium are necessary in the body, for example in enzyme based processes. Still further, zinc inhibits bacterial growth and reproduction, resulting in less production of odorous compounds by the bacteria. Preferably, skin protectants or skin protectors 170 are present in the deodorant composition 110 from about zero to twenty percent by weight.

### Herbal Extract

Herbal extracts are optionally incorporated in the deodorant composition 110 at a concentration of about zero to five percent by weight. Optional herbal extracts include but are not limited to chamomile and/or croton lechleri. Still additional optional herbal extracts include a tree extract of sangre de grado, which adds an appealing texture along with herbal benefits and/or dragon's blood, which is an anti-inflammatory that kills bacteria on skin.

### Preservatives

Preservatives 140 are optionally included in the deodorant composition 110 at a concentration of about zero to one percent by weight. Examples of preservatives 140 include methyl paraben, ethyl paraben, propyl paraben, potassium sulfate, and/or sodium benzoate.

### Cellulose

Forms of cellulose are optionally added to the deodorant composition 110. Cellulose acts to slow the release of the active ingredients or elements of the deodorant composition. Any form of cellulose is optionally used; however, preferred forms of cellulose include methyl cellulose, ethyl cellulose, and/or hydroxy propyl methyl cellulose.

### Additional Components

Ascorbic acid is another optional composition constituent, which performs as an anti-oxident. Ascorbic acid is a water soluble sugar acid with antioxidant properties. One form of ascorbic acid is commonly known as vitamin C. In another embodiment, ascorbic acid is optionally used with Vitamin A, as an anti-oxidant, in a composition used to treat body odor causing bacteria. Additional optional composition elements include each of xanthan gum and hydroxypropylmethyl cellulose, which perform as composition thickeners. Further, the xanthan gum performs as a rheology modifier. Still additional optional deodorant composition 110 ingredients include glyceryl laurate, stearic acid, sodium stearate, and/or a ricinoleate, such as glyceryl ricinoleate.

pH

The pH of skin is about 5.5. When the pH of the skin surface is less than about 3.8, skin peeling results. Additionally, side effects of skin adjusted to a pH of less than about 3.8 include: skin inflammation, dermatitis, itching, and/or sensitizing of the skin.

The inventor has determined that, in still yet another embodiment, a preferred pH range of the composition is in the range of about 4.0 to 6.0, which prevents the above described skin inflammation, dermatitis, itching, and/or sensitizing of the skin. A more preferred pH of the composition or formulation 110 for the treatment of odor forming bacteria is about 4.5, 5.0, or 5.5.

A preferred composition or formulation for the treatment of bacterially created components is provided in Table 1. The combination results in a pH in the range of 4.0 to 6.0. For example, the pKa of L-arginine of 12.48 adjusts the pH of the acetylsalicyclic acid up into the desired pH range of 4.0 to 6.0 and prevents the acid peeling resulting from the unbuffered and/or non pH adjusted acetylsalicyclic acid. Further, the application of a composition for the treatment of body odor with a pH of about skin does not disrupt normal skin functioning.

TABLE 1
Preferred Deodorant Formulation

| Ingredient | Percentage | Purpose |
|---|---|---|
| water | 60 to 64 | diluent |
| propylene glycol | 18 to 22 | skin moisturizer |
| acetylsalicyclic acid | 7 to 11 | active ingredient |
| L-arginine | 7 to 11 | active ingredient |

In another embodiment, a formulation or composition 110 for the treatment of body odor is provided. The combination results in a pH in the range of 4.0 to 6.0, includes a carrier 130, active ingredient 120, solubilizer, and skin moisturizer 150. The preferred formulation is provided in Table 1. However, each of the constituents present in Table 1 is optionally present in a range of concentrations. In another embodiment, the formulation contains: water in the range of about 20 to 99 percent, acetylsalicyclic acid in the range of about 0.1 to 40 percent, L-arginine in the range of about 0.1 to 40 percent, and propylene glycol in the range of about 0 to 40 percent, where percent is by either volume or mass. For example, the concentration of a first active ingredient of L-arginine is preferably about 9 percent; however a range of percentages of about 1, 2, 3, 4, 5, 6, 7, 8, 9, or 10 percent are acceptable and the concentration of a second active ingredient of acetylsalicyclic acid is preferably about 9 percent; however a range of percentages of about 0.5, 1, 2, 3, 4, 5, 6, 7, 8, 9, or 10 percent are acceptable, with the balance of the water and propylene glycol adjusted. The percentage of water is preferably about 59, 60, 61, 62, 63, 64, or 65 percent. The percentage of propylene glycol is preferably about 18, 19, 20, 21, or 22 percent.

In yet another embodiment, a formulation or composition for the treatment of and/or prevention of body odor is provided in Table 2.

TABLE 2
Formulation for treatment of bacterially created odorous by products

| Ingredient | Percentage |
|---|---|
| water | 20-99 |
| propylene glycol and/or solubilizer | 0 to 40 |
| acetylsalicyclic acid, salicyclic acid, and/or α-hydroxy acid | 0.1 to 40 |
| L-arginine, lysine, and/or glycine | 0.1 to 40 |
| pH | 4.5 to 6.0 |

In still yet another embodiment, a formulation or composition or formulation for the treatment of body odor is provided in Table 3. Percentages are optionally by mass or by volume. The formulation is optionally any combination of elements listed in Table 3 with a pH 4.0 to 6.0 or an adjusted pH of 4.0 to 6.0.

TABLE 3
Formulation for treatment of body odor causing bacteria

| Ingredient | Percentage |
|---|---|
| water and/or a diluent | 20 to 99 |
| aloe vera gel | 0 to 99 |
| propylene glycol and/or a moisturizer | 0 to 50 |
| acetylsalicyclic acid, salicyclic acid, and/or a α- | 0.1 to 40 |
| hydroxy acid | |
| lecithin | 0 to 20 |
| L-arginine, lysine, and/or glycine | 0.1 to 40 |
| lactic acid | 0 to 20 |
| shea butter | 0 to 20 |
| retinoic acid | 0 to 0.5 |
| ascorbic acid and/or an anti-oxidant | 0 to 5 |
| xanthan gum | 0 to 3 |
| hydroxypropylmethyl cellulose and/or a thickener | 0 to 3 |

In yet still another embodiment, the composition contains at least aspirin adjusted to a pH of about 4.0 to 6.0.

In still yet another embodiment, one or more of any of a skin moisturizer 150, preservative 140, herbal extract 160, and skin protectant 170 are optionally incorporated in any of the deodorant compositions 110 described herein.

### Method of Treatment

The formulation or composition is applied through a method of application of a thin veneer or spray of the formulation 110 to any of skin or a garment. Optionally, the composition is added to a bath.

In one embodiment, the composition 110 is added as a thin veneer or spray to skin, such as to an axillary region, under the arm, between the thighs, the leg, in the groin area, to a foot, and/or all over the body. The formulation is optionally applied with or without bathing and is optionally administered 1, 2, 3, or more times daily or every second, third or fourth day. Preferably, the deodorant 110 is applied to skin every twenty-four hours as more frequent applications are unnecessary for control of malodor. The formulation is optionally applied as a component of a cosmetically acceptable lotion, cream, ointment, soap, stick, solid, solid stick, gel, spray on, all body cream, suspension, or solution.

In another embodiment, the composition is added to a bath, a bath additive, a body soap, a body spray, and/or to a shower agent.

In yet another embodiment, the composition is used in treatment of fibrous material, such as clothing. Optionally, the composition is used in dry cleaning as an additive to a dry cleaning agent, as a localized treatment as a roll on, or as a spray.

Preferably, the formulation is sufficiently viscous to allow the formulation to remain on the affected area of the skin for a sufficient time period to reduce the body odor and/or to allow partial absorption of the composition into the skin.

In yet still another embodiment, any combination or permutation of any of the above described constituents is used to form a composition for the treatment of pseudofolliculitis barbae.

In still yet another embodiment, any combination or permutation of any of the above described constituents is used to form a formulation, composition, and/or mixture, cream, lather, foam, ointment, soap, liquid, or gel for application to the skin, bath, or in fiber treatment.

Although the invention has been described herein with reference to certain preferred embodiments, one skilled in the art will readily appreciate that other applications may be substituted for those set forth herein without departing from the spirit and scope of the present invention. Accordingly, the invention should only be limited by the Claims included below.

1. A composition for treatment and prevention of bromhidrosis, comprising:
an antimicrobial active ingredient comprising:
an amino acid; and
at least one of:
about one-tenth to forty percent by weight α-hydroxy acid;
about one-tenth to forty percent by weight salicyclic acid; and
about one-tenth to forty percent by weight acetyl salicyclic acid,
wherein the composition comprises a form of any of:
a spray;
a solid stick;
a soft solid;
a cream; and
a gel, and
wherein the composition comprises a component of any of:
a deodorant;
an antiperspirant;
a soap;
a bath additive;
a shower gel;
a skin cleaning product;
a garment cleaning product; and
a cleaning product.

2. The composition of claim 1, further comprising:
a base, wherein said composition comprises a pH of about 4.0 to 6.0.

3. The composition of claim 2, said amino acid comprising:
a guanidinium functional group having at least one pKₐ greater than 8.0.

4. The composition of claim 2, further comprising at least one of:
glucosamine;
n-acetylglucosamine; and
a form of glucosamine.

5. The composition of claim 2, said amino acid comprising at least one of:
about one-tenth to forty percent by weight arginine;
about one-tenth to forty percent by weight lysine;
about one-tenth to forty percent by weight histidine; and
about one-tenth to forty percent by weight glycine.

6. The composition of claim 5, further comprising at least two of:
a carrier;
a preservative;
a skin moisturizer;
an herbal extract; and
a skin protectant.

7. The composition of claim 6, wherein said preservative comprises at least one of:
about one-hundredth to one percent by weight methyl paraben;
about one-hundredth to one percent by weight ethyl paraben;
about one-hundredth to one percent by weight propyl paraben;
about one-hundredth to one percent by weight potassium sulfate;
about one-hundredth to one percent by benzyl alcohol;
about one-hundredth to one percent by weight phenoxyethanol;
about one-hundredth to one percent by weight sodium glycinate; and
about one-hundredth to one percent by weight sodium benzoate.

8. The composition of claim 6, wherein said skin moisturizer comprises at least one of:
about one-tenth to forty percent by weight aloe vera gel;
about one-tenth to forty percent by weight glycerin;
about one-tenth to forty percent by weight propylene glycol;
about one-tenth to forty percent by weight lecithin; and
about one-tenth to forty percent by weight shea butter.

9. The composition of claim 6, wherein said skin moisturizer comprises at least one of:
about one-tenth to forty percent by weight glycerin;
about one-tenth to forty percent by weight hyaluronan;
about one-tenth to forty percent by weight hyaluronic acid;
about one-tenth to forty percent by weight soybean oil;
about one-tenth to forty percent by weight coconut oil; and
about one-tenth to forty percent by weight palm oil.

10. The composition of claim 6, wherein said herbal extract comprises at least one of:
about one-hundredth to five percent by weight chamomile; and
about one-hundredth to five percent by weight croton lechleri.

11. The composition of claim 6, wherein said skin protectant comprises at least one of:
about one-tenth to twenty percent by weight a zinc salt; and
about one-tenth to twenty percent by weight a magnesium salt.

12. The composition of claim 6, wherein said skin protectant comprises:
about one-tenth to twenty percent by weight a form of silver.

13. The composition of claim 6, further comprising:
about one-hundredth to three percent by weight a form of cellulose.

14. The composition of claim 6, further comprising at least one of:
glyceryl laurate;
stearic acid;
sodium stearate; and
a ricinoleate.

15. A method for treatment of bromhidrosis, comprising:
providing a composition, comprising:
an antimicrobial active, comprising:
an amino acid; and
at least one of:
about one-tenth to forty percent by weight α-hydroxy acid;
about one-tenth to forty percent by weight salicyclic acid; and
about one-tenth to forty percent by weight acetyl salicyclic acid,
forming said composition as any of:
a spray;
a solid stick;
a soft solid;
a cream; and
a gel;
using the composition as any of:
a deodorant;
an antiperspirant;
a soap;
a bath additive;
a shower gel;
a skin cleaning product;
a garment cleaning product; and
a cleaning product; and
impeding bacterial action by applying said composition to an area comprising bacteria.

16. The method of claim 15, further comprising a step of:
providing at least one of:
a collagen; and
a protein; and
hydrolyzing in said composition at least one of said collagen and said protein to form said amino acid.

17. The method of claim 15, further comprising the step of:
treating an axillary region, said axillary region comprising at least one of:
propionobacteria; and
staphylococcus epidermidis bacteria.

18. The method of claim 15, further comprising the step of:
treating, using said composition, at least one of:
a garment;
bath water;
a fiber material;
a body;
hair; and
skin.

19. A composition for treatment and prevention of bromhidrosis, comprising:
an antimicrobial active, comprising at least two of:
an amino acid;
retinoic acid;
vitamin A;
an α-hydroxy acid;
salicyclic acid; and
acetyl salicyclic acid.
a base, wherein said composition comprises a pH of about 5.3 to 5.7,
wherein the composition comprises a form of any of:
a spray;
a solid stick;
a soft solid;
a cream; and
a gel, and
wherein the composition comprises a component of any of:
a deodorant;
an antiperspirant;
a soap;
a bath additive;
a shower gel;
a skin cleaning product;
a garment cleaning product; and
a cleaning product.
